(19) United States
(12) Patent Application Publication
(10) Pub. No.: US 2010/0063401 A1
(43) Pub. Date: Mar. 11, 2010

(54) ULTRASOUND ENDOSCOPE SYSTEM AND ULTRASOUND OBSERVATION METHOD

(75) Inventors: Kenichi NISHINA, Tokyo (JP); Masatoshi SATO, Yokohama-shi (JP); Takeharu NAKAZATO, Tokyo (JP); Sunao SATO, Yamato-shi (JP); Takuya IMAHASHI, Kawasaki-shi (JP); Shinichi TSUTAKI, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/207,150

(22) Filed: Sep. 9, 2008

(51) Int. Cl. A61B 8/14

(52) U.S. Cl. 600/466

(57) ABSTRACT

When an ultrasound endoscope arrives at an objective area, a puncture needle is located in a scan area of a first ultrasound image. Thereby, an image of the puncture needle is delineated on the first ultrasound image. Furthermore, an ultrasound probe is inserted into the puncture needle and an ultrasound transducer of the ultrasound probe is arranged in the objective area through the puncture needle. Then, the ultrasound probe is driven and a second ultrasound image is delineated. Detailed observation inside the objective area in which the puncture needle is punctured is possible with the second ultrasound image.

### BACKGROUND OF THE INVENTION

1. Field of the Invention

The present invention relates to an ultrasound endoscope system and an ultrasound observation method for observing an objective area under the guidance of the ultrasound endoscope.

2. Description of Related Art

Usually, under the guidance with an ultrasound endoscope, a lesion is punctured with a puncture needle inserted from a treatment instrument channel of the ultrasound endoscope, and a tissue is sucked and sampled from the lesion to diagnose pathologically. In addition, in recent years, by applying this procedure, for example, a drainage procedure which discharges a cyst fluid and the like which are stored in a lesion, such as a cyst of a pancreas, and also an injection procedure which injects a liquid medicine into an objective area, such as a lesion of a cancer, or nerve plexus has been performed.

However, reliable medical treatment may be unable to be performed only by such procedures using a puncture needle. For example, when a cyst of a pancreas gets worse and it becomes an abscess, its interior becomes solid necrosis and it may be unable to be discharged by the drainage procedure. In such a case, it is necessary to insert another comparatively large treatment instrument and to rake out the necrosis tissues from the abscess. In addition, depending on the case, it may become necessary to insert an endoscope into a lesion, and to remove the necrosis from the pancreatic abscess under the endoscopic observation. An operator needs to select a required procedure from these procedures.

In order to select such procedure, it is important to observe internal structure of a target site which is pierced with a puncture needle, in detail. In addition, as a matter of course, commonly to respective procedures, it is important to guide an ultrasound endoscope and a puncture needle to the objective area accurately.

### SUMMARY OF THE INVENTION

An ultrasound observation method according to one aspect of the present invention locates a puncture needle in a scan area of a first ultrasound image and delineates an image of the puncture needle on the first ultrasound image, inserts an ultrasound probe in the puncture needle, and drives the ultrasound probe to delineate a second ultrasound image.

In addition, an ultrasound endoscope system according to one aspect of the present invention comprises a first ultrasound observation unit which has a predetermined observation region, a first ultrasound image generating unit which can display a first ultrasound image on the basis of an observation result which is observed in the first ultrasound observation unit, a guide member with a needle-like distal end insertable and extractable to the observation region of the first ultrasound observation unit, a second ultrasound observation unit which has an external diameter insert-through-capable to the guide member, and a second ultrasound image generating unit which can display a second ultrasound image on the basis of an observation result which is observed by the second ultrasound observation unit.

Furthermore, an ultrasound endoscope system according to another aspect of the present invention comprises a first ultrasound observation unit which has a predetermined observation region, a protruding portion which is provided with predetermined positional relation to the first ultrasound observation unit and which protrudes by a predetermined amount, a puncture needle insertable and extractable to the predetermined observation region of the first ultrasound observation unit, an ultrasound probe which is insertable and extractable to the first ultrasound observation unit and the predetermined observation region, and which has an ultrasound observation surface in which the protruding portion can scan, and an ultrasound image generating unit which can display a first ultrasound image on the basis of an observation result of the first ultrasound observation unit, and a second ultrasound image on the basis of an observation result of the ultrasound probe.

Moreover, an ultrasound endoscope system according to another aspect of the present invention comprises a first ultrasound observation unit which is provided in a distal end surface of an insertion portion of an ultrasound endoscope and which has a predetermined observation region, a first treatment instrument channel through which a puncture needle is inserted, the first treatment instrument channel having a first opening in the distal end surface of the insertion portion, a second treatment instrument channel having a second opening in the distal end surface of the insertion portion, an ultrasound probe which is inserted through the second treatment instrument channel and which protrudes from the second opening, a protruding portion which protrudes from the distal end surface of the insertion portion to a scan area of the ultrasound probe, and an ultrasound image generating unit which can display a first ultrasound image on the basis of an observation result of the first ultrasound observation unit, and a second ultrasound image on the basis of an observation result of the ultrasound probe.

The above and other objects, features and advantages of the invention will become more clearly understood from the following description referring to the accompanying drawings.

### BRIEF DESCRIPTION OF THE DRAWINGS

FIG. 1 is an explanatory diagram showing an ultrasound endoscope system according to a first embodiment of the present invention;

FIGS. 2 and 3 are outline perspective views showing a distal end of the ultrasound endoscope;

FIG. 4 is a block diagram showing a configuration of a circuit unit which is provided in an ultrasound observation apparatus 6 and controls a rotational position of a radial image;

FIG. 5 is a perspective view showing a configuration of a puncture needle 5 in FIG. 1;

FIG. 6 is an explanatory diagram showing a configuration of a proximal end side of an ultrasound probe 71;

FIG. 7 is an explanatory diagram showing an outline sectional configuration of a distal end side of the ultrasound probe 71 in a state of being inserted through the puncture needle 5;

FIG. 8 is an explanatory diagram for describing connection between the ultrasound probe 71 and puncture needle 5;

FIG. 9 is an explanatory diagram for describing a procedure which uses the ultrasound endoscope;

FIG. 10 is an explanatory diagram showing a linear image and a radial image which are displayed on a display screen of a display unit 7:

FIGS. 11 to 13 are explanatory diagrams for describing a stylet:

FIG. 14 is a block diagram showing a circuit configuration of a toughness display unit;

FIGS. 15 and 16 are explanatory diagrams for describing positions of a toughness sensor 92 at the time of puncture;

FIG. 17 is an explanatory diagram showing a display example of toughness;

FIG. 18 is an explanatory diagram for describing a position of the toughness sensor 92;

FIG. 19 is an explanatory diagram showing another display example of toughness information;

FIG. 20 is a block diagram showing another circuit configuration of the toughness display unit;

FIG. 21 is an explanatory diagram for describing a state of a procedure of an embodiment;

FIGS. 22 and 23 are outline perspective views showing a modified example of the ultrasound endoscope;

FIG. 24 is an outline perspective view showing another modified example of the ultrasound endoscope;

FIGS. 25 and 26 are explanatory diagrams showing a modified example of the ultrasound probe inserted through a needle tube 54 of the puncture needle 5;

FIG. 27 is an explanatory diagram showing a modified example of the needle tube of the puncture needle through which the ultrasound probe is inserted;

FIG. 28 is an explanatory diagram showing another modified example of the needle tube of the puncture needle through which the ultrasound probe is inserted;

FIG. 29 is an explanatory diagram showing another modified example of the needle tube of the puncture needle through which the ultrasound probe is inserted;

FIGS. 30 and 31 are explanatory diagrams showing a second embodiment of the present invention;

FIG. 32 is an explanatory diagram showing an insertion shape of the ultrasound endoscope;

FIG. 33 is an explanatory diagram showing an ultrasound endoscope which has an insertion portion shape detection mechanism;

FIG. 34 is an explanatory diagram for describing arrangement of strain gages; and

FIG. 35 is an explanatory diagram showing the ultrasound endoscope which adopts another puncture needle.

### DETAILED DESCRIPTION OF PREFERRED EMBODIMENTS

Hereafter, with reference to drawings, embodiments of the present invention will be described in detail.

### First Embodiment

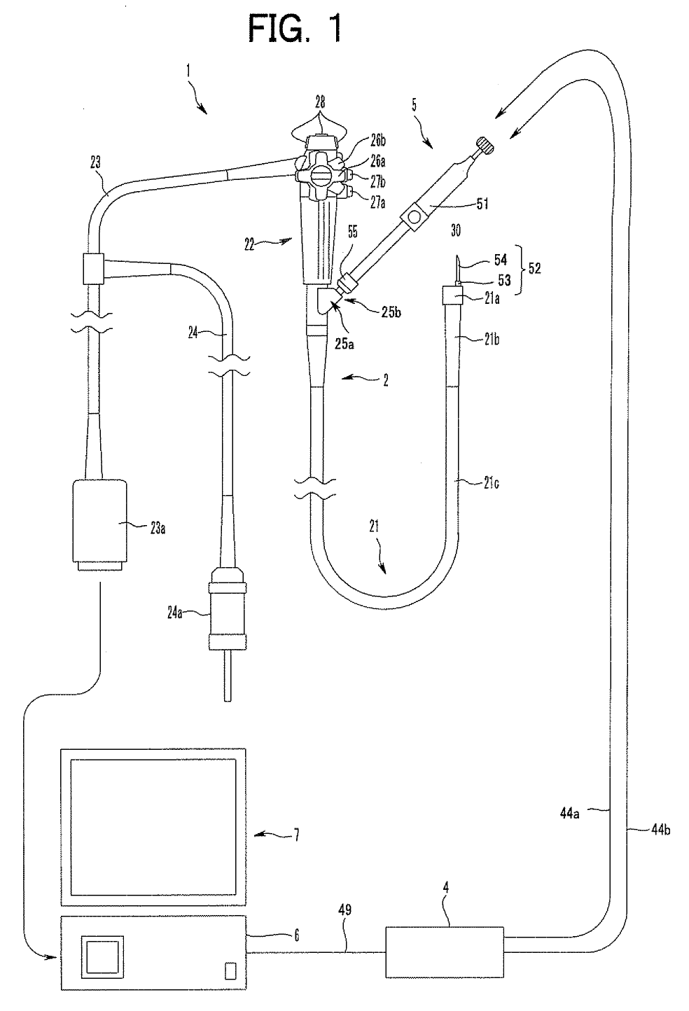

FIGS. 1 to 21 relate to a first embodiment of the present invention, and FIG. 1 is an explanatory diagram showing an ultrasound endoscope system according to the first embodiment of the present invention. In addition, hereafter, an ultrasound endoscope is abbreviated to an EUS.

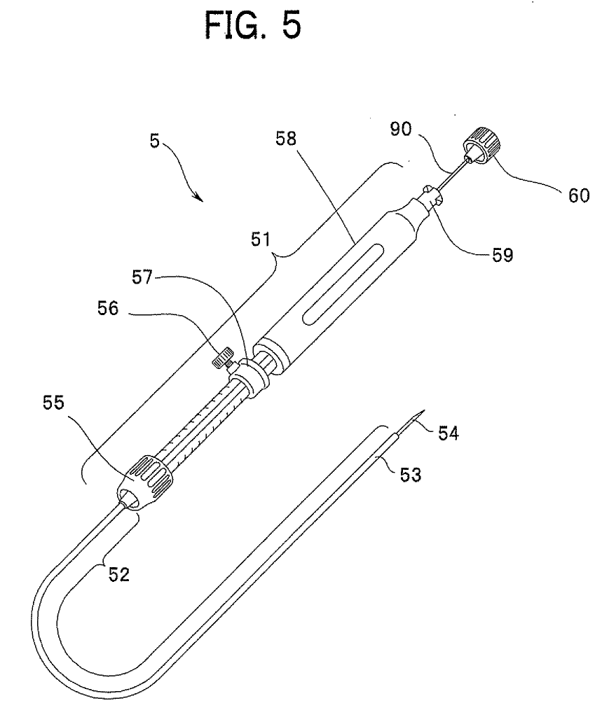

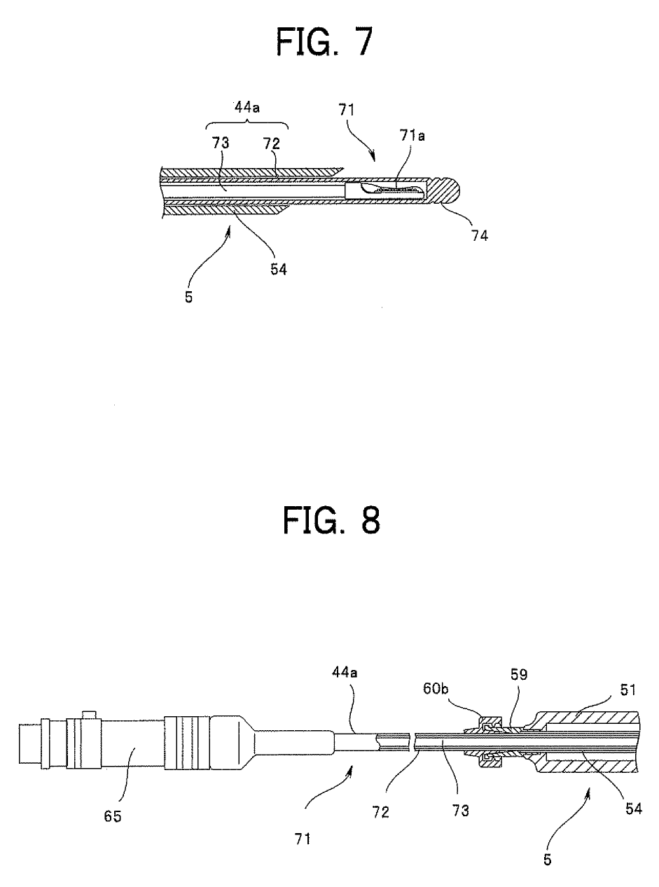

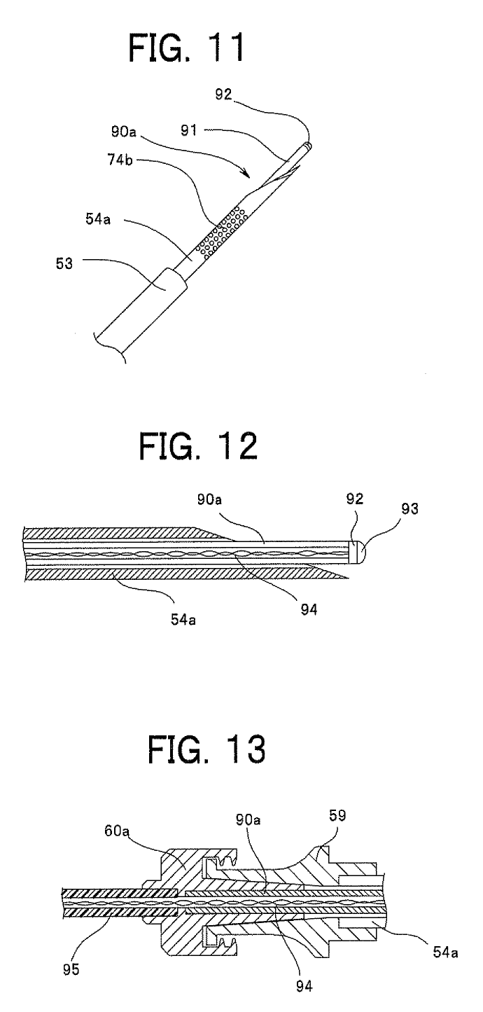

As shown in FIG. 1, an EUS system 1 of the present embodiment comprises an EUS 2 which is one of endoscopes, a puncture needle 5, an ultrasound observation apparatus 6, and a display unit 7. Furthermore, the EUS system 1 comprises an ultrasound probe 38 (refer to FIG. 3) provided insertably and extractably in a channel of the EUS 2, an ultrasound probe 71 (refer to FIG. 7) provided insertably and extractably in a needle tube of the puncture needle 5, and stylets 90 (refer to FIG. 5) and 90a (refer to FIG. 11) provided insertably and extractably in the needle tube of the puncture needle 5.

The EUS 2 mainly includes an insertion portion 21 inserted into an interior of a body, an operation portion 22 located in a proximal end of this insertion portion 21, a universal cord 23 extending from a side portion of this operation portion 22, and, for example, a cable 24 for a light source branched in a middle portion of this universal cord 23.

An ultrasound connector 23a which is detachable to the ultrasound observation apparatus 6 is provided in a proximal end portion of the universal cord 23. An endoscope connector 24a which is detachable to a light source device or a video processor apparatus which are not shown is provided in a proximal end portion of the cable 24 for a light source.

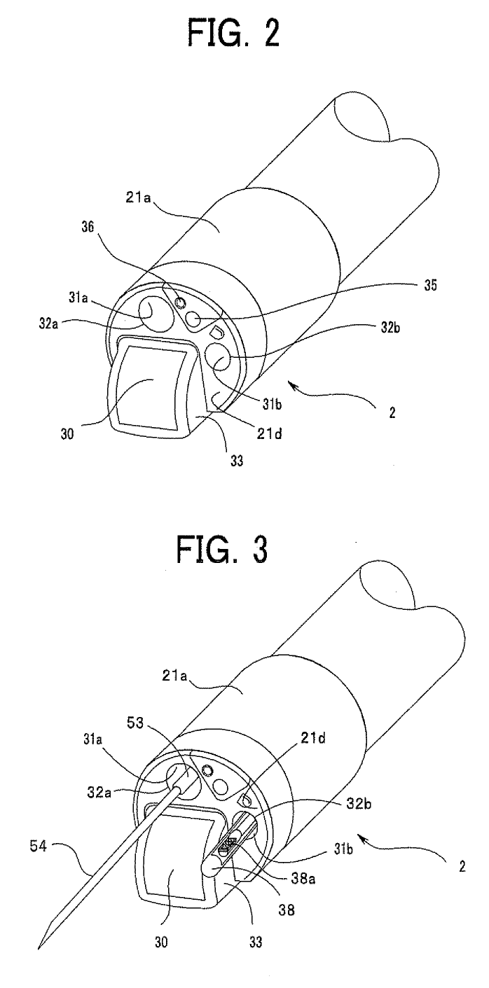

Treatment instrument insert-through ports 25a and 25b (the treatment instrument insert-through hole 25b is not shown) are provided in a distal end side of the operation portion 22. The treatment instrument insert-through ports 25a and 25b communicate with treatment instrument channels (refer to reference numerals 31a and 31b in FIG. 2) provided in the insertion portion 21, respectively.

The treatment instrument insert-through port 25a comprises a ferrule, and a securing ring 55 provided in a handle portion 51 of the puncture needle 5 and the like is connected to this ferrule. The securing ring 55 is detachable to the ferrule. And a needle tube 54 of the puncture needle 5 is inserted through the treatment instrument channel 31a through the treatment instrument insert-through hole 25a.

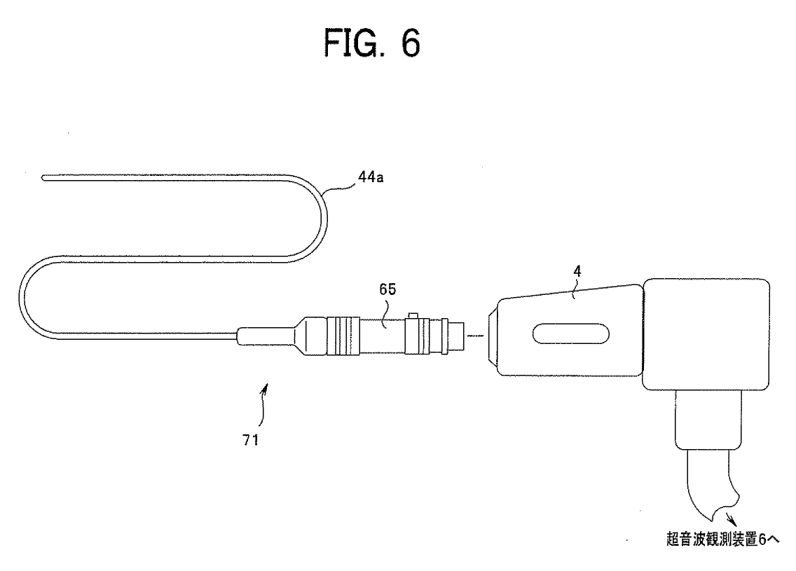

In the present embodiment, it is possible to insert the ultrasound probe 71 (refer to FIG. 7) and the like insertably and extractably through the needle tube 54. As mentioned later, the ultrasound probe 71 has an ultrasound transducer 71a in a distal end, and has a transfer unit 44a in a proximal end side. The transfer unit 44a is connected to a driving unit 4 through an ultrasound connector 65 (refer to FIG. 6), and the ultrasound probe 71 is driven by the driving unit 4. The driving unit 4 can transmit an echo signal from the ultrasound probe 71 to the ultrasound observation apparatus 6 through a cable 49.

In addition, it is possible to insert the ultrasound probe 38 (refer to FIG. 3) through the treatment instrument channel 31b through the treatment instrument insert-through hole 25b. As mentioned later, the ultrasound probe 38 has an ultrasound transducer 38a in a distal end, and has a transfer unit 44b in a proximal end side. The transfer unit 44b is connected to the driving unit 4 through an ultrasound connector not shown (the same as the connector 65 in FIG. 6), and the ultrasound probe 38 is driven by the driving unit 4. The driving unit 4 can transmit an echo signal from the ultrasound probe 38 to the ultrasound observation apparatus 6 through the cable 49. In addition, it is desirable to set an ultrasound frequency by the ultrasound transducer 38a, and an ultrasound frequency by the ultrasound transducer 30 to be frequencies which are mutually different.

Furthermore, although the echo signal from the EUS 2 and ultrasound probe 38 is transmitted to the ultrasound observation apparatus 6 in the present embodiment, it is also good to provide two ultrasound observation apparatuses 6 to transmit the echo signal of the EUS 2 to one ultrasound observation apparatus 6, and to transmit the echo signal of the ultrasound probe 38 and ultrasound probe 71 to another ultrasound observation apparatus.

Reference numerals 26a and 26b denote bending operation knobs, reference numeral 27a does an air supply/water supply button, reference numeral 27b does a suction button, and reference numeral 28 does a switch. The switch 28 performs, for example, a display change of the display unit 7, a freeze instruction of a display image, or a release instruction, a start/stop instruction of toughness measurement by a toughness sensor mentioned later, or the like.

The insertion portion 21 provides consecutively a distal end rigid portion 21a, a bending portion 21b, and a flexible tube portion 21c sequentially from a distal end side. The bending portion 21b is configured so as to bend actively in up and down, right and left directions, for example, by an operation of the bending operation knobs 26a and 26b. The flexible tube portion 21c has flexibility.

FIGS. 2 and 3 are outline perspective views showing a distal end of the EUS.

The treatment instrument channels 31a and 31b have distal end openings 32a and 32b respectively in a distal end surface 21d of the distal end rigid portion 21a. The treatment instrument channel 31a is arranged so that a central axis near the distal end opening 32a may approximately coincide with an ultrasound scan surface by the ultrasound transducer 30, and a treatment instrument which performs a puncture and the like can be inserted therethrough. In addition, an objective optical system 35 and an illumination optical system 36 are provided in a distal end surface 21d of a distal end rigid portion 21a.

An electronic scanning-type ultrasound transducer 30 is arranged in the distal end side of the distal end rigid portion 21a. The ultrasound transducer 30 is a convex array for example, and includes a plurality of ultrasound elements being arranged inside. The EUS 2 obtains an echo signal by the ultrasound transducer 30 transmitting and receiving an ultrasound with switching the respective ultrasound elements. The echo signal from the ultrasound transducer 30 is transmitted to the ultrasound observation apparatus 6 through the ultrasound connector 23a. On the basis of the echo signal from the ultrasound transducer 30, an ultrasound image (linear image) which has a section parallel to an insertion axis of the insertion portion 21 is obtained.

In the present embodiment, a structure protruded comparatively greatly from the distal end surface 21d is not provided between the distal end openings 32a and 32b. Thereby, it becomes possible to delineate the needle tube 54 with the ultrasound probe 38 in the case of inserting the puncture needle 5 through the treatment instrument channel 31a to protrude the needle tube 54 from the distal end opening 32a, and inserting the ultrasound probe 38 through the treatment instrument channel 31b to protrude the ultrasound transducer 38a, which is provided in the distal end of the ultrasound probe 38, from the distal end opening 32b.

The ultrasound transducer 38a of the ultrasound probe 38 is freely rotatable with centering on the insertion axis of the ultrasound probe 38 almost parallel to the insertion axis of the insertion portion 21. The ultrasound probe 38 obtains an echo signal by transmitting and receiving an ultrasound while the ultrasound transducer 38a rotates. The echo signal from the ultrasound transducer 38a is transmitted to the ultrasound observation apparatus 6 through the ultrasound connector and driving unit 4 which are not shown, and an ultrasound image (radial image) of a section which goes directly to the insertion axis of the insertion portion 21 is obtained on the basis of the echo signal.

In addition, in the present embodiment, the ultrasound transducer 30 has a protruding portion 33 protruding from the distal end rigid portion 21a. Thereby, the protruding portion 33 is delineated by the ultrasound probe 38. In addition, the protruding portion 33 is provided in a position except on a line linearly connecting the distal end openings 32a and 32b mutually. In addition, in order that ultrasound observation of the protruding portion 33 may become easy, it is preferable to give ultrasound reflection processing to a surface of the protruding portion 33.

For example, as the ultrasound reflection processing, concavo-convex processing treatments, such as sand blasting process, satin finish processing, and dimple processing treatment, the coating treatment of a resin containing bubbles or metal powder, and the like are conceivable.

The echo signal from the ultrasound transducer 30 is inputted into the ultrasound observation apparatus 6 through the ultrasound connector 23a, and the echo signal from the ultrasound probe 38 or 71 is inputted through the cable 49. The ultrasound observation apparatus 6 can display a linear image on the basis of an output of the ultrasound transducer 30, and a radial image on the basis of outputs of the ultrasound probes 38 and 71 on a display screen of the display unit 7.

A radial image from the ultrasound probe 38 has an unfixed reference position of a rotary direction, and a vertical direction of a radial image which is displayed and a vertical direction of a distal end surface 21d of the insertion portion 21 do not correspond. The ultrasound observation apparatus 6 can display a radial image in an arbitrary rotational position by controlling, for example, writing and reading of a radial image with respect to memory for a display.

In the present embodiment, it is possible to perform display with making the vertical direction of a radial image correspond to the vertical direction of the distal end surface 21d using an ultrasound image of the protruding portion 33.

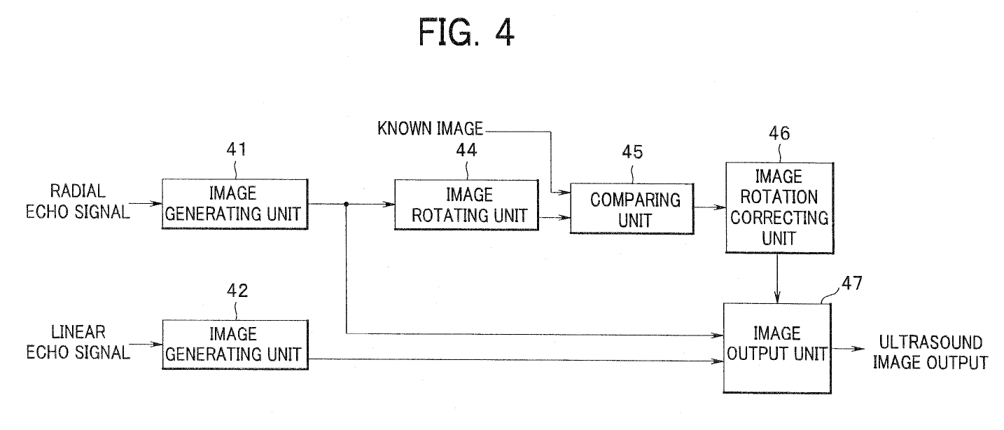

FIG. 4 is a block diagram showing a configuration of a circuit unit which is provided in the ultrasound observation apparatus 6 and controls a rotational position of a radial image.

An echo signal from the ultrasound transducer 38a or an echo signal from the ultrasound transducer 30 is inputted into the image generating units 41 and 42, respectively. In addition, the ultrasound probe 38 can delineate at least the protruding portion 33 protruded from the distal end opening 32a. The image generating units 41 and 42 generate and output a radial image or a linear image, which is a two-dimensional image, on the basis of the inputted echo signals.

The radial image and linear image from the image generating units 41 and 42 are inputted into the image output unit 47. While storing the inputted image, the image output unit 47 performs image synthesis and an output in order to make the linear image and radial image displayed on a common display screen.

On the other hand, the radial image from the image generating unit 41 is inputted also into an image rotating unit 44. The image rotating unit 44 rotates the inputted radial image suitably, and outputs the radial image after rotation, and information on its rotating amount to a comparing unit 45. As for the delineated protruding portion 33 included in the radial image, a position, based on the vertical direction of the distal end surface 21d of the insertion portion 21, and a shape are known. A known image about the delineated protruding portion 33 is stored in the comparing unit 45, and the comparing unit 45 compares the radial image from the image rotating unit 44, and the known image.

When detecting by an image matching method that the known image of the protruding portion 33 coincides with a part of the radial image, the comparing unit 45 outputs the information on the rotating amount of the radial image in this case to an image rotation correcting unit 46.

The image rotation correcting unit 46 controls an output of the radial image from the image output unit 47 on the basis of the information on the rotating amount which is inputted from the comparing unit 45, and makes the vertical direction of the radial image coincide with the vertical direction of the distal end surface 21d of the insertion portion 21. Since the vertical direction of a linear image coincides with the vertical direction of the distal end surface 21d of the insertion portion 21, an ultrasound image where vertical directions of the radial image and linear image coincide is displayed on the display unit 7.

In addition, since the protruding portion 33 includes the ultrasound transducer 30, mutual positional relation is known. It is also sufficient that the image output unit 47 may obtain a position of a linear scan surface from a position of the delineated protruding portion 33 to display a line (linear scan line display) which shows the position of the linear image on the radial image.

Furthermore, it is also possible that the image output unit 47 switches display ranges of the linear image and radial image, which are displayed on a display screen, with interlocking them.

Moreover, it is also sufficient to automate rotation of the radial image by the circuit in FIG. 4, or it is also sufficient that an operator rotates the radial image manually with referring to the linear image and radial image.

FIG. 5 is a perspective view showing a configuration of a puncture needle 5 in FIG. 1.

As shown in FIGS. 5 and 3, the puncture needle 5 is configured by comprising the handle portion 51 and a channel insertion portion 52, and the channel insertion portion 52 is configured by comprising a sheath 53 and the needle tube 54. The channel insertion portion 52 is inserted through the treatment instrument channel 31a from the treatment instrument insert-through port 25a, and is configured protrudably from the distal end opening 32a shown in FIG. 3.

The handle portion 51 is configured by arranging, for example, a securing ring 55, an adjuster knob 56, a needle adjuster 57, a needle slider 58, a suction ferrule 59, and a stylet cap 60 sequentially from a distal end side.

The needle tube 54 is arranged with being inserted through the sheath 53 retractably. This needle tube 54 is formed of, for example, a metal pipe, such as a stainless steel pipe or a nickel titanium pipe. A sharp-shaped cutting portion is formed in a distal end (hereinafter, this is also called a needle point) of the needle tube 54.

The stylet 90 or the stylet 90a which is inserted through the needle tube 54 is connected to the stylet cap 60, and the stylet cap 60 is connected to the suction ferrule 59. A proximal end portion of the needle tube 54 is fixed in one piece to the suction ferrule 59 by adhesion and the like.

The needle adjuster 57 is slide-fixed or released by the adjuster knob 56. By loosening the adjuster knob 56 to release fixation of the needle adjuster 57, it becomes possible to make a needle slider 8 slide. In addition, a protrusion length of the needle tube 54 from the distal end of the sheath 53 is adjusted by adjusting suitably a distance between the fixed positions of a needle slider 8 and needle adjuster 57.

FIG. 6 is an explanatory diagram showing a configuration of a proximal end side of the ultrasound probe 71, and FIG. 7 is an explanatory diagram showing an outline section configuration of a distal end side of the ultrasound probe 71.

In the present embodiment, the puncture needle 5 also has a function as a guide member which guides the ultrasound probe 71 to the distal end of the needle tube 54. As the puncture needle 5, for example, what has about φ0.6 mm to φ1.2 mm of inner diameter of the needle tube 54 is used. In addition, as the ultrasound probe 71, for example, what has about φ0.5 to φ1 mm of external diameter and 15 to 30 MHz of ultrasound frequency is used.

The transfer unit 44a of the ultrasound probe 71 is connected to the driving unit 4 through the ultrasound connector 65 in a proximal end side, as shown in FIG. 6. As mentioned above, the driving unit 4 is connected to the ultrasound observation apparatus 6.

As shown in FIG. 7, the transfer unit 44a includes a shaft 73 and a sheath 72, and the shaft 73 connects the ultrasound transducer 71a and a motor which is provided in the driving unit 4 and which is not shown. An outer periphery of the shaft 73 is covered with the sheath 72.

The ultrasound transducer 71a is electrically connected to the driving unit 4 by a wiring which is inserted through the shaft 73 and which is not shown. With this wiring, a high voltage pulse signal for ultrasound wave generation from the driving unit 4 is supplied to the ultrasound transducer 71a. The ultrasound transducer 71a receives an ultrasound reflected by a living body tissue with performing electric-sound conversion of this high voltage pulse signal and transmitting the ultrasound for observation, performs acousto-electric transformation of the received ultrasound, and transmits it to the driving unit 4 through a wiring as an electric signal.

As shown in FIG. 7, the ultrasound probe 71 is inserted through the needle tube 54 to a position where the ultrasound transducer 71a in the distal end protrudes from the needle tube 54 of the puncture needle 5. By transmitting and receiving an ultrasound with rotating the ultrasound transducer 71a by a motor in this state centering on the insertion axis of the needle tube 54, the ultrasound probe 71 can acquire a radial image in front of the distal end of the needle tube 54.

That is, in the present embodiment, even if it is a site where the insertion portion 21 of the EUS 2 cannot be inserted, so long as it is a site where the puncture needle 5 can be punctured, it is possible to perform observation by an ultrasound radial image.

Furthermore, as for the ultrasound probe 71, the ultrasound reflection unit 74 is formed in a distal end. The ultrasound reflection unit 74 is given ultrasound reflection processing. As the ultrasound reflection processing, known methods, such as dimple processing and sand blasting, can be adopted. For example, what forms many small holes in stainless steel is also sufficient as ultrasound reflection processing. In addition, it is also sufficient to provide the same ultrasound reflection unit near the distal end portion of the needle tube 54.

FIG. 8 is an explanatory diagram for describing connection between the ultrasound probe 71 and puncture needle 5.

As shown in FIG. 8, as for the proximal end side of the ultrasound probe 71, the ultrasound connector 65 is provided in the proximal end portion of the transfer unit 44a, and the transfer unit 44a is connected to the driving unit 4 by this ultrasound connector 65. The shaft 73 is covered with the sheath 72. A ferrule 60b provided in the sheath 72 is connected to the suction ferrule 59 of the handle portion 51 of the puncture needle 5. Connecting structure of the suction ferrule 59 is made into the Luer connector.

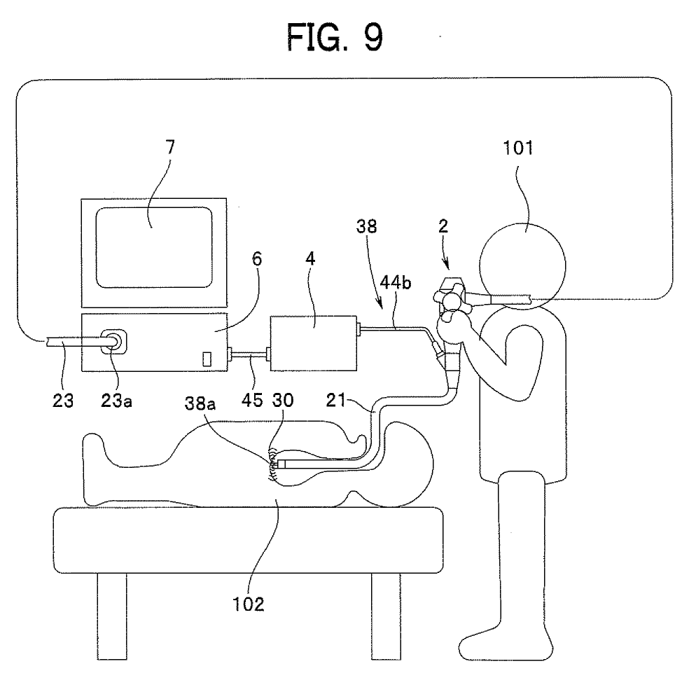

Next, the various procedures using the EUS system configured in this way will be described with reference to FIGS. 9 to 21.

Heretofore, it is known to perform medical treatment procedures, such as an EUS-FNA (EUS-guided fine needle aspiration), a drainage procedure, an injection procedure, using an EUS which mounts a linear/convex type ultrasound transducer. The EUS is configured such that its scan surface may become in parallel to the insertion axis of an endoscope. In such a system, since an ultrasound transducer delineates a section parallel to the insertion axis of the endoscope, an ultrasound image changes a lot just by turning slightly an insertion portion around the shaft. For example, when a needle is bent and separates from a scan surface when puncturing is performed, it is necessary to shake a distal end of an endoscope to relook at the needle, and hence, experience is required for an operation and a procedure takes time.

By using not only a linear image parallel to the insertion axis of the EUS, but also a radial image which has a section orthogonal to the insertion axis, the present embodiment makes it possible to perform quickly and securely medical treatment procedures, such as the EUS-FNA, and EUS-guided drainage procedure and injection procedure.

FIG. 9 is an explanatory diagram for describing a procedure which uses the EUS.

As shown in FIG. 9, an operator 101 inserts the insertion portion 21 of the EUS 2 into a body through, for example, a mouth of a patient 102, observes an endoscope image displayed on the display unit 7, and inserts the ultrasound transducer 30 to near an objective area. Then, the operator contacts the ultrasound transducer 30 to a luminal wall.

### (Procedure to Puncture Using Linear Image and Radial Image)

The operator performs a linear scan, a convex scan, or a sector scanning with the ultrasound transducer 30 provided in the distal end of the EUS 2, and obtains an ultrasound image (linear image) of a section parallel to the insertion axis of the insertion portion 21.

Next, the operator inserts the ultrasound probe 38 into the treatment instrument channel 31b of the EUS 2, and protrudes the distal end portion of the ultrasound probe 38 from the distal end opening 32b by a length approximately comparable to protruding quantity of the protruding portion 33 of the ultrasound transducer 30.

The operator performs a radial scan with rotating the ultrasound transducer 38a of the ultrasound probe 38, and obtains an ultrasound image (radial image) of a section which goes directly to a distal end of the insertion portion 21.

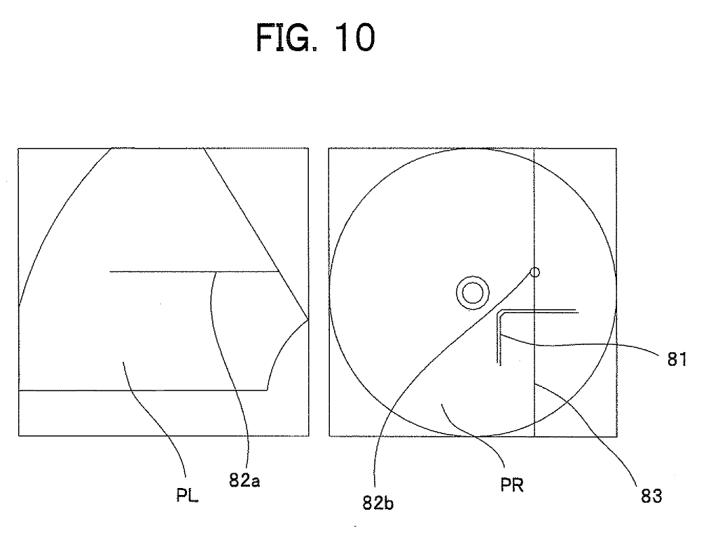

FIG. 10 is an explanatory diagram showing a linear image and a radial image which are displayed on a display screen of the display unit 7. On the display screen of the display unit 7, a linear image PL is displayed in a left side and a radial image PR is displayed in a right side. Both of a linear-shaped image 82a in the linear image PL, and a round image 82b in the radial image PR are the needle tube 54 of the puncture needle 5 delineated. In addition, an L-shaped image 81 in the radial image PR is the delineated protruding portion 33 delineated by the ultrasound probe 38. In addition, in the radial image PR, a linear-shaped linear scan line display 83 which shows a position (scan area) of a linear image is also displayed.

In addition, the protruding portion 33 is given the ultrasound reflection processing and is securely delineated by the ultrasound probe 38. In addition, since the ultrasound transducer 30 and ultrasound probe 38 have different ultrasound frequencies, an artifact by mutual ultrasounds emitted by both, and the like do not arise.

The ultrasound observation apparatus 6 grasps positional relation between the radial image and endoscope (image of linear image) using an image of the protruding portion 33 in the radial image PR, and displays the radial image whose positional relation is made to coincide with a vertical direction of the distal end surface 21d (linear image). In addition, an operator may perform this rotation of a radial image manually.

First, the operator 101 operates the EUS 2, guides the distal end portion of the insertion portion 21 to near an objective area, and delineates the objective area in the linear image PL with the ultrasound transducer 30. At this time, the operator confirms the objective area by the radial image PR, guides the distal end portion of the insertion portion 21 to the vicinity of the objective area, and makes a delineated objective area displayed in the linear image PL.

Next, the operator 101 inserts treatment instruments, such as the puncture needle 5, through the treatment instrument channel 31a of the EUS 2, and performs an EUS-guided treatment.

When a treatment instrument (needle tube 54 of the puncture needle 5, and the like) bends, or when the insertion portion 21 rotates, the treatment instrument may be located out of a delineated range of the linear image. Also in this case, in the present embodiment, it is possible to return a rotary direction of the insertion portion 21 simply by confirming the position of the image of the treatment instrument in the radial image PR.

Thus, it is possible to find the objective area simply by searching the objective area with both the linear image and radial image. In addition, even when a treatment instrument (needle or the like) bends and an image based on the treatment instrument separated from the linear image, it is possible to confirm by a radial image to where and how far it separates, and it is possible to guide the insertion portion 21 easily to a position where the treatment instrument can be again delineated on the linear image. Thereby, while a burden of an operator is eased, time of a procedure is shortened and mitigation of a patient's pain can be also achieved.

### (Procedure at the Time of a Puncture Using Toughness Sensor)

By the way, depending on a site where the needle tube 54 of the puncture needle 5 is punctured, advanced skill and prolonged working hours may be needed for an operation of advancing the needle tube 54. For example, heretofore, an EUS-FNI (EUS-guided fine needle injection) procedure of puncturing a hypodermic needle in an objective area under EUS guidance using an EUS which mounts a linear convex type ultrasound transducer, and injects a medicine and the like through a needle tube is known. In this procedure, it is necessary to puncture in an organ (nerve or the like) which is located just before an organ, without puncturing an organ such as a blood vessel, and to inject a liquid medicine. That is, a needle tip must be located just before a blood vessel, an ultrasound image must be observed carefully, and a needle must be carried forward carefully. Hence, because of always operating a distal end of an EUS to confirm that the needle tip is delineated in an ultrasound scan area, and relying on touch of feeling with fingers at the time of carrying forward a needle, and the like, operator's skill is needed for operations.

Then, in the present embodiment, a method of performing procedures simply without requiring skill by using a toughness sensor is proposed. FIGS. 11 to 20 are for describing the method of position confirmation of a needle tip using a toughness sensor.

FIGS. 11 to 13 are explanatory diagrams for describing a stylet.

As a stylet which is made to be inserted through a needle tube of the puncture needle 5, a stylet 90a shown in FIG. 11 to FIG. 13 is adopted. In addition, a needle tube 54a is adopted as the needle tube of the puncture needle 5. The needle tube 54a is different from the needle tube 54 in that an ultrasound reflection portion 74b is provided in a distal end of the needle tube which is exposed from the sheath 53. In addition, when a position of the needle tip of the needle tube 54a does not need to be delineated, the needle tube 54 can be adopted.

The stylet 90a is a hollow tube made from a nickel titanium alloy (Ni—Ti), and is inserted through the needle tube 54a to be extended to the needle tip, and its distal end portion 91 can be exposed in front of the needle tip of the needle tube 54a. The toughness sensor 92 is provided in a distal end of the stylet 90a. The toughness sensor 92 detects toughness of a body tissue by being pressed on the body tissue. For example, as the toughness sensor 92, it is possible to adopt what includes an ultrasound piezoelectric element and acquires toughness information of tissue by a change of a resonance frequency. For example, such a sensor is described in detail in Japanese Patent Application Laid-Open Publication No. 8-261915, Japanese Patent Application Laid-Open Publication No. 9-285439, Japanese Patent Application Laid-Open Publication No. 7-270261, and the like.

As shown in FIG. 12, a distal end forming portion 93 made from resin or rubber may be provided in a distal end side of the toughness sensor 92 if needed.

A signal from the toughness sensor 92 is transmitted through a distribution cable 94 for a sensor. This distribution cable 94 for a sensor is arranged in a space inside the stylet 90a.

As shown in FIG. 13, a proximal end side of the stylet 90a is fixedly installed in a stylet cap 60a. The stylet 90a is mounted on the puncture needle 5 by the stylet cap 60a being fixed to a proximal end portion of the suction ferrule 59 of the puncture needle 5. In addition, a Luer connector is adopted as structure of the suction ferrule 59. A cable 95 connected to a processor apparatus 98 (refer to FIG. 14) for a sensor is mounted in the stylet cap 60a, and the distribution cable 94 for a sensor is arranged inside the stylet 90a and the cable 95, and connects the toughness sensor 92 and processor apparatus 98 for a sensor.

FIG. 14 is a block diagram showing a circuit configuration of a toughness display unit.

In FIG. 14, a signal from the toughness sensor 92 is inputted into the processor apparatus 98 for a sensor through the distribution cable 94 for a sensor. Based on the signal from the toughness sensor 92, the processor apparatus 98 for a sensor obtains information (toughness information) with regard to toughness of a body tissue, and outputs it to the ultrasound observation apparatus 6. The ultrasound observation apparatus 6 can display data showing the toughness of the body tissue on the display screen of the display unit 7 based on the inputted toughness information.

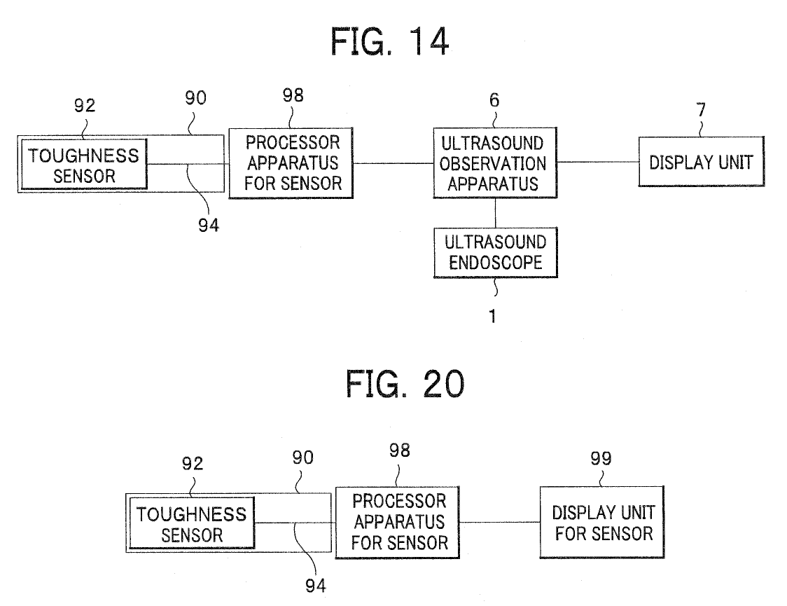

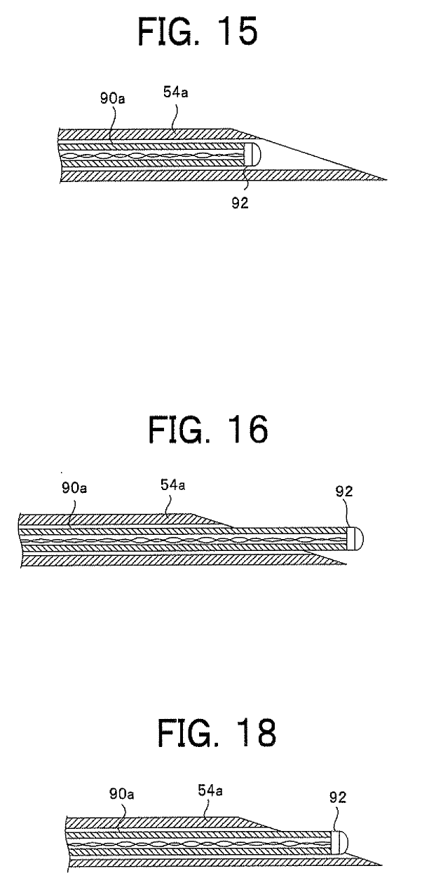

Next, an EUS-guided puncture method using the puncture needle 5 through which such the stylet 90a is inserted will be described with reference to FIGS. 15 to 19.

FIGS. 15 and 16 are explanatory diagrams for describing positions of the toughness sensor 92 at the time of puncture.

First, the stylet cap 60a is removed from the suction ferrule 59, and the toughness sensor 92 in the distal end of the stylet 90a is made to be contained in the needle tube 54a, as shown in FIG. 15. Thereby, a smooth puncture becomes possible by the sharp needle tip. When it is punctured to near an objective area, the stylet cap 60a is fixed to the suction ferrule 59. Thereby, the stylet 90a is pushed into the needle tube 54a, and as shown in FIG. 16, the stylet 90a protrudes by fixed quantity from the needle tip of the needle tube 54a. Thereby, the toughness sensor 92 abuts on a target tissue, an output according to the toughness of the objective area is transmitted to the processor apparatus 98 for a sensor through the distribution cable 94 for a sensor from the toughness sensor 92.

In addition, positional relation (length relation) between the stylet 90a and needle tube 54a is made into the extent of the toughness sensor 92 in the distal end of the stylet 90a protruding a little (1 mm or less) than the needle tip when the stylet cap 60a is thoroughly fixed to the suction ferrule 59.

Furthermore, it is possible to instruct ON/OFF of toughness measurement by an operator's simple operation by assigning the function to a foot switch which is not shown, or the switch 28 provided in the EUS 2. In this case, ON/OFF information on the switch of toughness measurement is also inputted into the ultrasound observation apparatus 6 with toughness information.

The processor apparatus 98 for a sensor calculates the information on toughness from an output or a change of an output of the toughness sensor 92, and outputs the toughness information to the ultrasound observation apparatus 6. The ultrasound observation apparatus 6 makes data (for example, a numerical value, a graph, etc.) based on the toughness information displayed in an ultrasound image on the display screen of the display unit 7.

In addition, it is also sufficient to provide a needle tip detection portion, which detects a position of the ultrasound reflection portion 74b provided in the distal end of the needle tube 54a and which is not shown, in the ultrasound observation apparatus 6. For example, on the basis of known information, including the length data of the ultrasound reflection portion 74b, a puncture angle of the needle tube 54a, and the like, the needle tip detection portion extracts a highly-intensive linear delineated image from an ultrasound image, and recognizes it as the delineated needle tube 54a with an image matching method with known information. On the basis of the recognized delineated needle tube 54a, a position of the needle tube 54a on the ultrasound image is obtained.

FIG. 17 is an explanatory diagram showing a display example in this case. In the example of FIG. 17, toughness data 113 is displayed below an ultrasound image 111. An image 112 corresponding to the ultrasound reflection portion 74b of the needle tube 54a is delineated in the ultrasound image 111. The toughness data 113 express information based on a detection result of the toughness sensor 92 in a numerical value, a graph, or the like. The toughness data 113 in FIG. 17 shows magnitude of toughness in a bar graph, and toughness is expressed by a change of a rate of an area shown by presence of hatching in FIG. 17.

Hereafter, similarly, toughness of a tissue is confirmed each time by the method which advances the needle tube 54a and protrudes the stylet 90a, and which is shown in FIGS. 15 to 17. An operator can confirm the toughness of a tissue with advancing the needle tube 54a, and can grasp that the needle tip of the needle tube 54a arrives, for example, just before a blood vessel wall and the like, because of a change of the toughness.

Furthermore, it is also possible to confirm the toughness of a tissue simultaneously with advancing the needle tube 54a by setting positional relation between the needle tube 54a and stylet 90a suitably.

FIG. 18 is an explanatory diagram for describing a position of the toughness sensor 92 in this case.

That is, although the toughness sensor 92 in the distal end of the stylet 90a protrudes from a distal end opening of the needle tube 54a in a state that the stylet cap 60a is fixed to the suction ferrule 59 thoroughly as shown in FIG. 18, the positional relation between the stylet 90a and needle tube 54a is set to be in a state that it does not protruded from the needle tip.

Since the needle tip protrudes, a puncture to a tissue is possible, and, since the toughness sensor 92 is also exposed, toughness measurement is also possible. Thereby, it is possible to measure the toughness continuously with advancing the needle tube 54a.

In addition, the ultrasound observation apparatus 6 may have memory which associates and stores the position of the needle tip, which are detected by the needle tip detection portion, and the toughness information, and which is not shown. The ultrasound observation apparatus 6 may comprise a graphical display function which displays a graph which shows toughness in a location corresponding to a position of the needle tip based on the information stored in the memory.

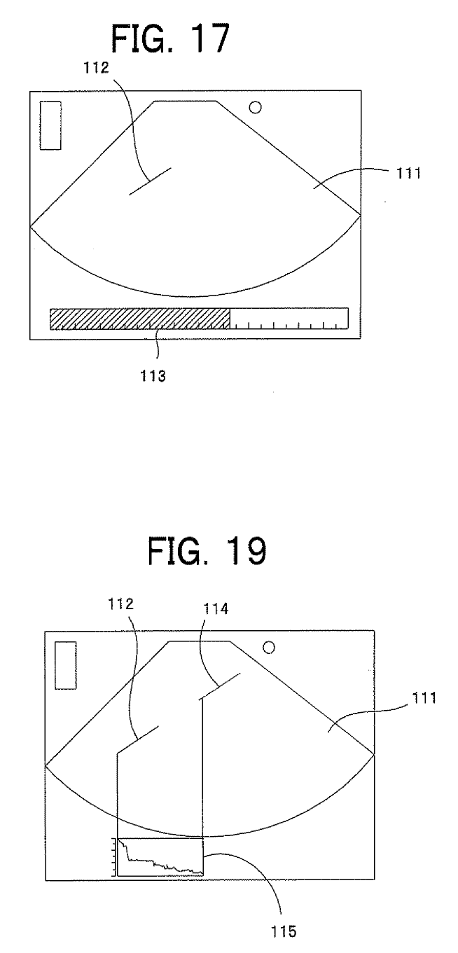

FIG. 19 is an explanatory diagram showing another display example of toughness data.

FIG. 19 displays toughness in real time. That is, toughness measurement is started when an operator operates a foot switch or the like. The ultrasound observation apparatus 6 sequentially stores the position of the needle tip and the toughness information at that time. The ultrasound observation apparatus 6 makes the ultrasound image 111 and toughness data 115 displayed on a display screen on the basis of the stored information. The toughness data 115 display the measurement result of the toughness in real time in a numerical value, a graph, or the like.

In an example of FIG. 19, the toughness data 115 is displayed below the ultrasound image 111. Starting position display 114 which shows a position of the needle tube 54a at the time of a measurement start is also displayed on the ultrasound image 111 besides the image 112 corresponding to the needle tube 54a. The toughness data 115 in FIG. 19 shows the measurement of toughness in a line graph, a horizontal axis corresponds to the position of the needle tube 54a, and a vertical axis corresponds to the toughness. That is, whenever the needle tube 54a advances, the toughness in the position is displayed in real time perpendicularly below a position of the needle tip of the needle tube 54a.

In addition, it is also sufficient to detect it by the needle tip detection portion when the needle tip of the needle tube 54a is retreating, and to make the graph not updated.

By referring to the toughness data 115, an operator can easily grasp a change of the positional relation between the toughness information and needle tip, that is, a change of organization structure inside the objective area more intuitively. Thereby, it is possible to achieve further reduction of an operators load.

In addition, in the state of FIG. 18, it is also conceivable that a puncture is difficult depending on the toughness of a tissue.

However, there is a small gap between an outer peripheral surface of the stylet 90a and the inner peripheral surface of the needle tube 54a. For this reason, when temporarily piercing into a very hard tissue, the stylet 90a which has elasticity moves in a zigzag direction within the needle tube 54a, and the toughness sensor 92 is pushed into the needle tube 54a by the tissue. Thereby, it becomes in the same state as that in FIG. 15, and a puncture is possible also in the hard tissue.

FIG. 20 is a block diagram showing another circuit configuration of the toughness display unit. An example in FIG. 20 adopts a display unit 99 for a sensor which displays measurement result of toughness independently.

Thus, with inserting a stylet, which has a toughness sensor in a distal end, into a needle tube, measurement of the toughness of a tissue is performed with advancing the needle tube. Thereby, it becomes possible to measure the toughness of an objective area quantitatively, and it becomes possible to judge objectively that the needle tip hits a blood vessel wall, for example. Hence, it becomes also possible that those who are not skilled in a method perform a procedure in a level equivalent to a skilled person. For example, it becomes possible that an unskilled doctor also performs comparatively easily a procedure of advancing a needle tip just before a celiac artery, and performing ethanol infusion, in the case of a celiac plexus block.

In addition, although toughness is measured so as to prevent the needle tip from advancing to a blood vessel or the like unnecessarily in the description mentioned above, it is available also for a purpose of detecting that the needle tip has advanced securely into an objective area in the present embodiment. For example, in pathological changes, such as a cyst, elasticity of an adventitia may be high and an interior may be a liquid, and in such a case, the adventitia is pushed and just depressed by the needle tip of the needle tube 54a, but the needle tip may not encroach on the cyst. In this case, it is not possible to judge only in a position of the needle tip whether the needle tip has encroached in the objective area, such as a cyst. However, since a change of the toughness is measured in the present embodiment, when a value of the toughness became small rapidly, it is also possible to grasp that the needle tip has encroached in the objective area.

### (Procedure After Puncture Using Ultrasound Probe in Needle Tube)

By the way, heretofore, medical treatment procedures, such as the EUS-FNA, drainage procedure, and injection procedure, and a diagnostic procedure which performs ultrasound observation of a pancreaticobiliary area using an EUS from a stomach or a duodenum are known.

As for an EUS used in such procedure, a comparatively low ultrasound frequency of, for example, 5 to 12 MHz and the like is adopted because of a request of hoping to perform ultrasound observation to a comparatively deep area in many cases. However, in such a comparatively low ultrasound frequency, detailed structure inside an objective area is unobservable.

In addition, heretofore, although a diagnostic procedure (intraductal ultrasonography; IDUS) which inserts an ultrasound probe into a pancreatic duct and a bile duct for a transduodenal papilla target and performs ultrasound observation has been known, this IDUS might be unable to be given to an example in which cannulation to duodenal papilla is difficult. In addition, even if the cannulation can be performed and the IDUS is possible, X-ray observation is needed for position confirmation of a probe which is inserted into the transduodenal papilla target, and there is a possibility of X-rays exposure.

In the present embodiment, also in such a case, detailed structure observation of an interior of the objective area is possible. That is, in the present embodiment, after an EUS-guided puncture is performed, the stylet 90a of the puncture needle 5 is extracted, and the ultrasound probe 71 (refer to FIG. 7) is inserted into the needle tube 54 of the puncture needle 5.

In this case, it is confirmed that a distal end of the ultrasound probe 71 in the needle tube 54 protrudes by a suitable distance from the needle tip of the needle tube 54 with an ultrasound image obtained by the EUS 2. That is, protruding quantity of the ultrasound probe 71 is confirmed so that the ultrasound transducer 71a may protrude from the needle tube 54.

In addition, the ultrasound connector 65 (refer to FIG. 6) is connected to the driving unit 4. Then, an ultrasound scan is made with rotating the ultrasound transducer 71a of the ultrasound probe 71 by the driving unit 4.

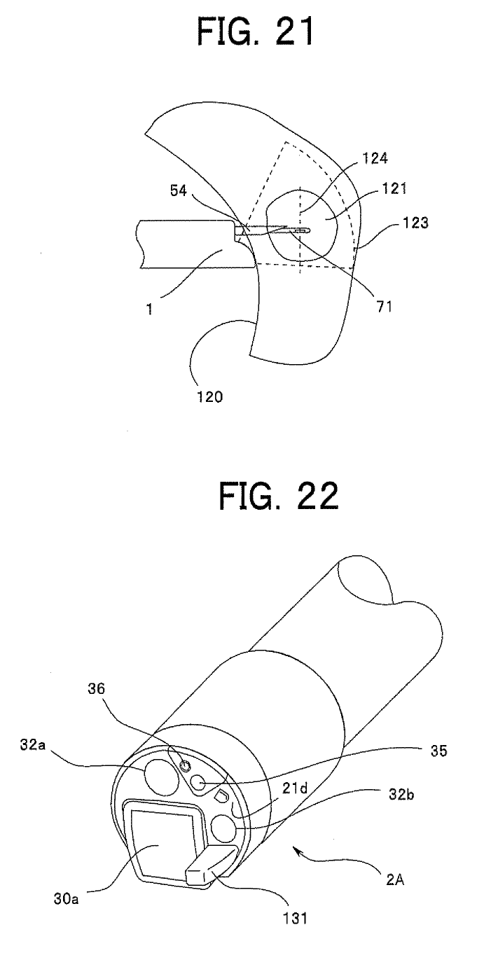

FIG. 21 is an explanatory diagram showing this state. The distal end of the insertion portion 21 of the EUS 2 is made to contact with a luminal wall 120 of a stomach, a duodenum, or the like. Then, the needle tube 54 is punctured into an objective area 121. A range 123 shown by doted lines is a scan area of an ultrasound image by the ultrasound transducer 30 (refer to FIG. 2) of the EUS 2. In addition, a dotted line 124 shows a scan area of an ultrasound image by the ultrasound transducer 71a of the ultrasound probe 71.

It is possible to delineate an interior of the objective area 121, into which the needle tube 54 is punctured, in detail by making the ultrasound transducer 71a of the ultrasound probe 71 protrude from the distal end of the needle tube 54, and performing an ultrasound scan. That is, an ultrasound image can be obtained from a place close to the objective area 121 by inserting the ultrasound probe 71 through the needle tube 54 after an EUS-guided puncture into the objective area 121.

That is, since the ultrasound probe 71 approaches the objective area 121 and just picks up an ultrasound image, it is possible to use a sufficiently high ultrasound frequency. That is, as for the ultrasound probe 71, it is possible to use an ultrasound frequency higher than that of the EUS 2, and hence, it is possible to obtain an ultrasound image with a higher resolution.

Thereby, it becomes possible to grasp more detailed structure inside the objective area 121, such as a run state of blood vessels not more than φ1 mm which cannot be delineated with the ultrasound transducer 30, and existence of a tubercle with height of 2 mm or less.

When the ultrasound scan by the ultrasound probe 71 is completed, the ultrasound probe 71 is extracted from the needle tube 54. Subsequently, various procedures are performed according to observation result with an ultrasound observation image. For example, required treatments, such as suction sampling of a tissue or a cell from the needle tube 54, injection of a liquid medicine, and insertion of a guide wire, are performed consecutively.

Thus, in the present embodiment, the EUS-guided puncture is performed. Then, after the EUS-guided puncture, the ultrasound probe is inserted into a needle tube of a puncture needle, after it is confirmed with the ultrasound observation by the EUS that a distal end of the ultrasound probe has reached the interior of the objective area, the ultrasound probe is made to scan, and then, the ultrasound image from the interior of the objective area is obtained. In this way, an interior structure of an objective area is observable in detail. Since it is possible to grasp detailed structure inside an objective area before an operation, it becomes possible to perform a subsequent treatment properly. In addition, it is possible to achieve reduction of an operator's load.

For example, it is possible to observe the interior of the objective area in a position which is comparatively separated from a stomach or a duodenum in detail, to make the ultrasound probe arrive at the objective area even when the objective area is a pathological change of a bile duct or a pancreatic duct and cannulation is difficult, and to observe detailed internal structure. Furthermore, it is also possible to observe in detail a disease or a structure which exists in a pancreas and gall area.

In addition, it is possible to grasp, for example, presence of a minute blood vessel before an operation of necrosis removal. When existence of the minute blood vessel is confirmed, the necrosis removal is performed after first solidifying the blood vessel by ethanol infusion or the like. Thereby, time to spend on a hemostasis operation to bleeding caused by the necrosis removal without confirming the presence of small blood vessels, which leads to reduction of an operator's load.

Furthermore, at the time of a diagnosis of an intrapancreatic bile duct papillary mucinous neoplasm (IPMN), it is possible to confirm existence of a minute tubercle which cannot be delineated with the ultrasound transducer of the EUS by performing puncture to the pathological change and confirming the presence of a lesser tubercle, and to perform a more reliable diagnosis.

In addition, since it becomes possible to perform position confirmation of the ultrasound probe by ultrasound observation by the ultrasound transducer of the EUS, it is possible to eliminate or reduce X-ray exposure.

Furthermore, although procedure of performing puncture to the objective area, thereafter extracting the stylet 90a from the needle tube 54 of the puncture needle 5, and inserting the ultrasound probe 71 into the needle tube 54 is used here, it is also satisfactory to insert beforehand the ultrasound probe 71 into the needle tube 54 before performing the puncture.

### MODIFIED EXAMPLE

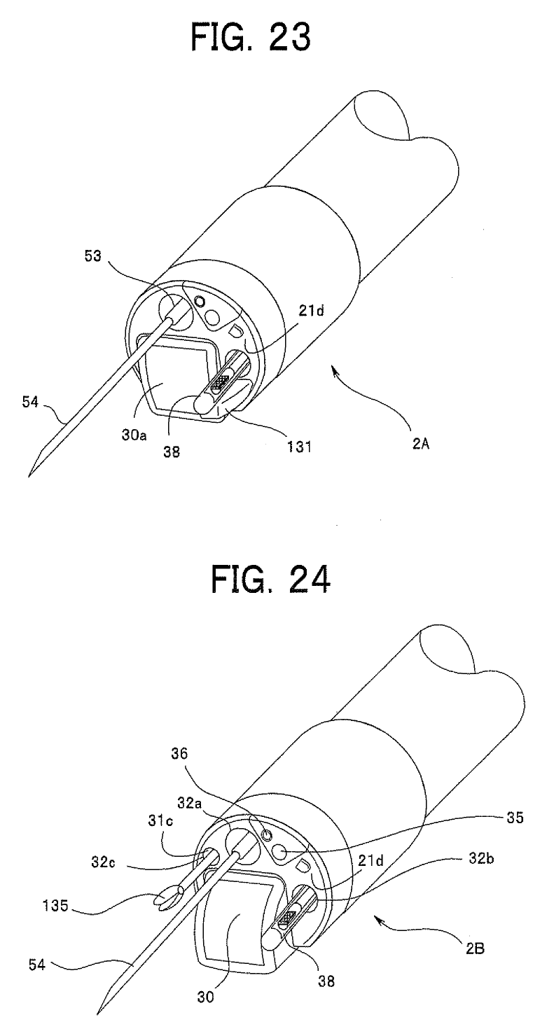

FIGS. 22 and 23 are outline perspective views showing a modified example of the EUS.

An EUS 2A in FIGS. 22 and 23 differs from the EUS 2 in FIGS. 2 and 3 in that not only an ultrasound transducer 30a is used in place of the ultrasound transducer 30, but also a protruding portion 131 is provided.

The ultrasound transducer 30a of the EUS 2A in FIGS. 22 and 23 has a surface approximately parallel to the distal end surface 21d, and its protruding quantity from the distal end surface 21d is very small. Hence, the protruding portion 33 does not exist in the ultrasound transducer 30a.

On the other hand, the protruding portion 131 is provided in the EUS 2A similarly to the protruding portion 33. Thereby, the protruding portion 131 is delineated by the ultrasound probe 38. The protruding portion 131 is provided in a position except on a line linearly connecting the distal end openings 32a and 32b mutually. In addition, in order that ultrasound observation of the protruding portion 131 may become easy, ultrasound reflection processing is given to a surface of the protruding portion 131.

In addition, as the ultrasound reflection processing, concavo-convex processing treatments, such as sand blasting process, satin finish processing, and dimple processing treatment, the coating treatment of a resin containing bubbles or metal powder, and the like are conceivable.

Also in such a modified example configured, the protruding portion 131 is delineated in the radial image obtained by the ultrasound probe 38. A position of the protruding portion 131 in the EUS 2A is known, and it is possible to make a vertical direction of the radial image coincide with a vertical direction of a linear image automatically by an image of the protruding portion 131 delineated in the radial image.

FIG. 24 is an outline perspective view showing another modified example of the EUS.

The EUS 2B in FIG. 24 differs from the EUS 2 in FIG. 3 in respect of including three treatment instrument channels. The treatment instrument channel 31c has a distal end opening 32c in the distal end surface 21d.

A treatment instrument such as a grasping forceps 135 can be inserted through the treatment instrument channel 31c. In addition, it is also possible to feed plain water or air using the treatment instrument channel 31c.

According to such a configuration, it is possible to insert the grasping forceps 135 to the treatment instrument channel 31c, and to hold a luminal wall during a scan of a linear image and a radial image. Thereby, the insertion portion 21 of the EUS 2B is stabilized and delicate position and attitude control of the distal end of an endoscope become possible.

In addition, it becomes possible to feed an ultrasound delineation medium, such as plain water or an ultrasound jelly, through the treatment instrument channel 31c. Since the plain water and ultrasound jelly for transmitting an ultrasound can be additionally supplied with keeping a state that the radial image is delineated, even when a bubble appears near the distal end of the endoscope and hinders transfer of the ultrasound, it becomes possible to remove the bubble quickly and to obtain a good ultrasound image.

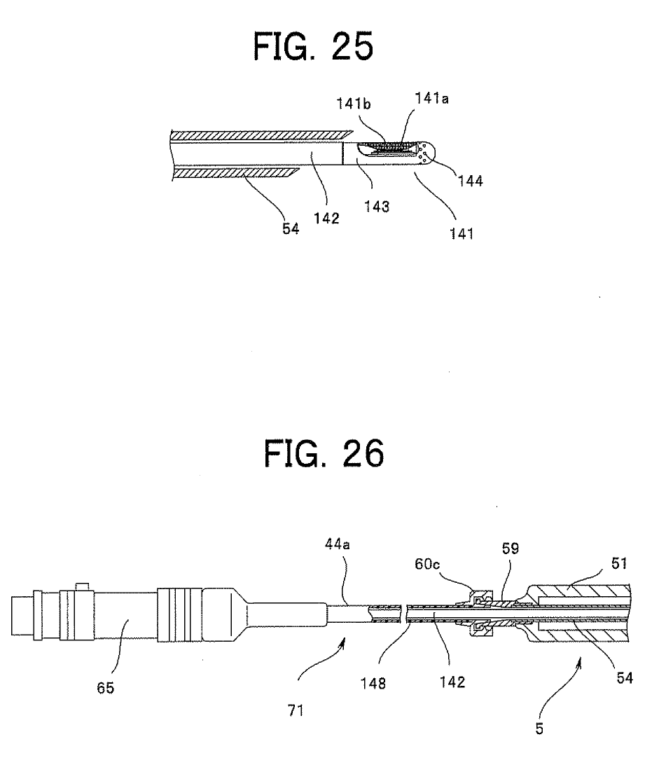

FIGS. 25 and 26 are explanatory diagrams showing a modified example of the ultrasound probe inserted through the needle tube 54 of the puncture needle 5.

Unlike the ultrasound probe 71 in FIG. 7, an ultrasound probe 141 in FIG. 25 is not covered with a sheath. An ultrasound transducer 141a is provided in a distal end of the ultrasound probe 141, and the ultrasound transducer 141a has structure of being held by a housing 143. An ultrasound reflection unit 144 is provided in a distal end side of the housing 143. The ultrasound reflection unit 144 is given ultrasound reflection processing. As the ultrasound reflection processing, for example, a known method, such as dimple processing or sand blasting, is used.

The housing 143 is fixed to a shaft 142 in the proximal end side, and the shaft 142 is connected to the driving unit 4 in FIG. 1 and transmits a turning force to the housing 143. The shaft 142 is a hollow layer coil, wiring which is not shown is arranged therein, and the driving unit 4 and ultrasound transducer 141a are electrically connected by this wiring.

An acoustic radiation surface of the ultrasound transducer 141a is filled with a material which transmits an ultrasound, for example, a filler material 141b, such as polymethylpentene or polyethylene, and the whole housing 143 including the ultrasound transducer 141a is formed with this filler material 141b so as to have an approximately cylindrical side face.

As shown in FIG. 26, the ultrasound connector 65 is provided in the proximal end side of the ultrasound probe 141, which is connected to the driving unit 4 by this ultrasound connector 65. A sheath 148 covers the shaft 142 from the ultrasound connector 65 to the handle portion 51 of the puncture needle 5, and a ferrule 60c provided in a distal end of the sheath 148 is connected to the suction ferrule 59 of the handle portion 51. Connecting structure of the suction ferrule 59 is made into the Luer connector.

In addition, although not shown, when a three-way stopcock or a T-tube is provided between the ferrule 60c and suction ferrule 59, it becomes possible to inject a medium through a gap between the needle tube 54 and shaft 142.

Furthermore, it is also sufficient to provide a sliding mechanism in the proximal end side of the sheath 148, or the like to make it possible to change a length from the ultrasound connector 65 to the ferrule 60c.

Since the sheath of the ultrasound probe 141 is omitted according to such a modified example, it becomes possible to use an ultrasound probe with the thinner needle tube 54. When the thinner needle tube 54 becomes usable, even when an EUS-guided puncture is difficult with a thick needle tube, it is possible to perform a puncture comparatively easily.

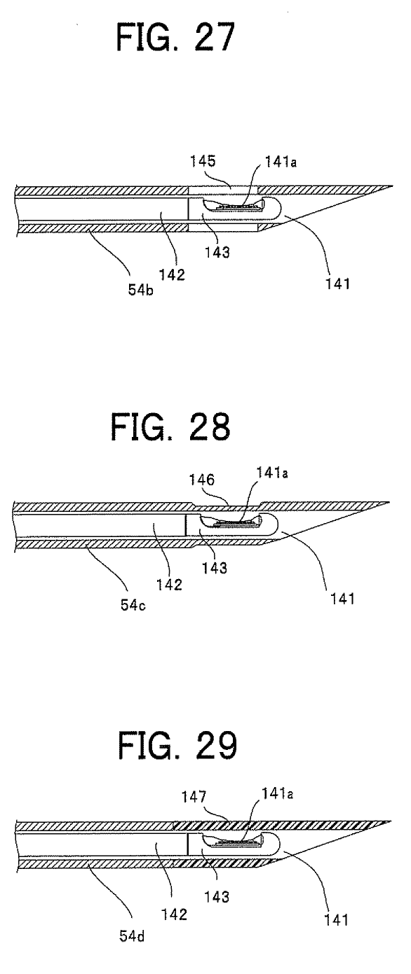

FIG. 27 is an explanatory diagram showing a modified example of the needle tube of the puncture needle through which the ultrasound probe is made to be inserted.

The example of FIG. 27 adopts the needle tube 54b instead of the needle tube 54 in FIG. 25. In the example in FIG. 27, as for the ultrasound probe 141, a portion of the housing 143 holding the ultrasound transducer 141a is arranged inside the needle tube 54b. In the needle tube 54b, a plurality of slits 145 is provided in a position facing the housing 143.

According to such a configuration, the ultrasound probe 141 is inserted so that the ultrasound transducer 141a may face the slit 145 of the needle tube 54b. When the ultrasound probe 141 performs an ultrasound scan in this state, a part of the ultrasound emitted from the ultrasound transducer 141a is transmitted into an objective area through the slit 145, and a part of a reflected ultrasound is received by the ultrasound transducer 141a through the slit 145. In this way, it is possible to obtain an ultrasound image also in this modified example.

According to this modified example, since there is no necessity of protruding the ultrasound probe 141 from the needle tube 54b even if an interior of an objective area is not a liquid but a solid tissue, an ultrasound scan becomes possible.

FIG. 28 is an explanatory diagram showing another modified example of the needle tube of the puncture needle through which the ultrasound probe is inserted.

An example in FIG. 28 adopts the needle tube 54c instead of the needle tube 54b in FIG. 27. In the needle tube 54b, a thin wall portion 146 is formed in a position facing the housing 143. The thin wall portion 146 easily transmits an ultrasound. Other configurations and operations and effects are the same as those of the modified example in FIG. 27.

FIG. 29 is an explanatory diagram showing another modified example of the needle tube of the puncture needle through which the ultrasound probe is made to be inserted.

An example in FIG. 29 adopts a needle tube 54d instead of the needle tube 54b in FIG. 27. The needle tube 54d includes a resin (e.g., polyether ether ketone (PEEK) or the like) into which a metal braid or a coil is impregnated, and a distal end side from a portion of a distal end of the needle tube which faces the ultrasound transducer 141a includes only the resin 147. The resin 147 easily transmits an ultrasound.

Other configurations and operations and effects are the same as those of the modified example in FIG. 27.

### Second Embodiment

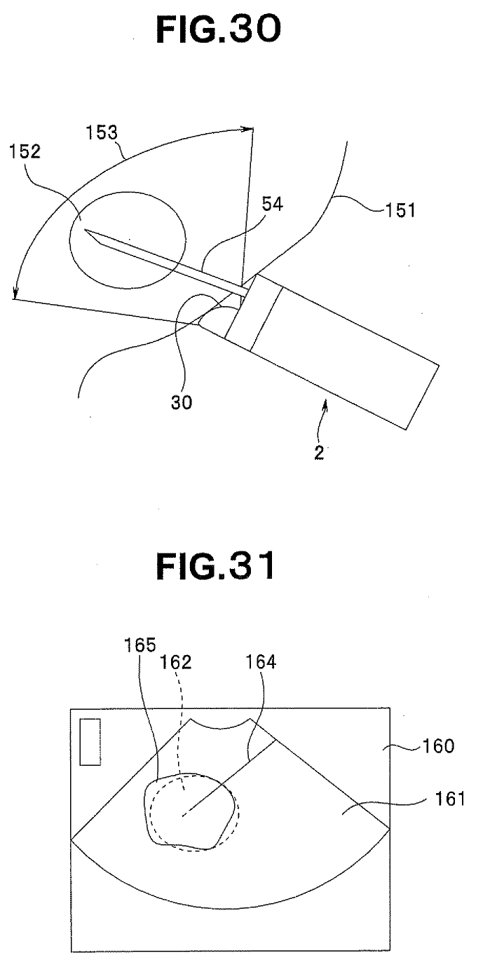

FIGS. 30 and 31 are explanatory diagrams showing a second embodiment of the present invention.

### (Injection Using Ultrasound Contrast Agent)

The present embodiment facilitates observation in the case of performing injection by the puncture needle 5 after a puncture. For example, there is an EUS-guided celiac plexus block as a pain relaxation therapy of a terminal pancreatic cancer. In order to paralyze or destroy a nerve plexus, ethanol is injected into celiac plexus through a needle which is punctured under EUS guide. Nevertheless, it is hard to see the injected ethanol on an ultrasound image. For this reason, it was difficult to confirm whether the injected ethanol was spread to a desired area.

In the present embodiment, what contains an ultrasound contrast agent as a medicine to be injected is adopted. As the ultrasound contrast agent, there are Definity (registered trademark) (Bristol-Myers Squibb), Sonazoid (registered trademark), and the like.

An operator contacts the ultrasound transducer 30 of the EUS 2 with a luminal wall 151 as shown in FIG. 30 using the method of the first embodiment. Then, an objective area 152, such as a nerve plexus, is caught in a desired position in an ultrasound scan area 153. When doing so, as shown in FIG. 31, an image 162 of the objective area 152 is delineated in an ultrasound image 161 on a display screen 160 of the display unit 7.

In addition, on the ultrasound image 161, an image 164 corresponding to the needle tube 54 of the puncture needle 5 is also delineated. When confirming that the image 162 is displayed in the desired position on the ultrasound image 161, and that the distal end of the needle tube 54 is located in the objective area 152 with the image 164, an operator injects a liquid medicine, which contains an ultrasound contrast agent, through the needle tube 54.

The liquid medicine injected from the needle tube 54 is diffused from a needle tip, and widens in the objective area 152. Since containing the ultrasound contrast agent, this liquid medicine is delineated on the ultrasound image 161 as the image 165 of the liquid medicine as shown in FIG. 31. Thereby, the operator can observe a situation of the injected liquid medicine easily.

In addition, not only when injecting a liquid medicine into a celiac plexus, but also when injecting a liquid medicine into other locations, this can be applied similarly. For example, this is applicable also to an injection of a liquid medicine into a pancreatic cyst and the like. In addition, the medicine is not limited to ethanol. It is also applicable to an injection of an anticancer drug or a gene for a medical treatment of a pancreatic cancer, and the like.

Thus, in the present embodiment, since a liquid medicine to be injected is made to contain an ultrasound contrast agent, the situation of the injected liquid medicine is observable on an ultrasound image. An operator can confirm the injection state and an injection range of the medicine with an ultrasound image, and can perform various medical treatments safely and efficiently.

### (Observation of Insertion Shape of EUS Before Puncture)

By the way, in an EUS-guided puncture, because a distal end of the EUS may move by a reaction force by the puncture, depending on a condition, for an ultrasound image to become difficult to be seen, much time may be spent on the puncture. For example, depending on an insertion shape of the EUS, a distal end portion of the EUS retreats because it cannot bear a reaction force of a tissue at the time of a puncture, an operation for correcting this is needed, and much time may be spent on this operation.

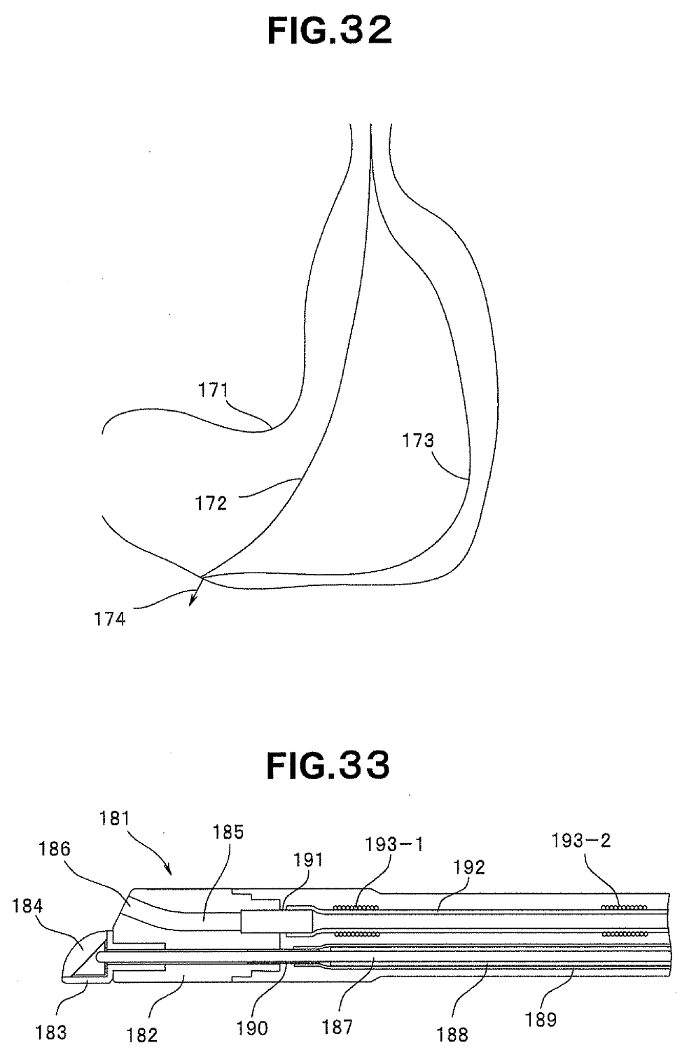

FIG. 32 is an explanatory diagram showing an insertion shape of the EUS in this case. FIG. 32 shows an example of inserting the EUS into a stomach 171. An insertion portion shape 172 is in a direction approximately parallel to a puncture direction 174 to a stomach wall. On the other hand, an insertion portion shape 173 has a large angle between an insertion direction and a puncture direction 174 to a stomach wall in a puncture position. That is, in the state of the insertion portion shape 173, the distal end portion of the EUS may retreat by a reaction of the stomach wall, and an operation for correcting this may be needed.

Then, an operator confirms a shape of the insertion portion 21 of the EUS 2 three-dimensionally, after making an objective area delineated by ultrasound observation of the EUS 2 (refer to FIG. 1). In addition, a magnetic sensor system can be used as means to confirm a shape of the insertion portion of the EUS. The magnetic sensor system is described in full detail in Japanese Patent Application Laid-Open Publication No. 9-28662, Japanese Patent Application Laid-Open Publication No. 2001-46318, and the like.

That is, first, at a first step, an operator inserts an EUS into a luminal portion suitable for delineating an objective area, starts an ultrasound scan, and delineates the objective area with an ultrasound image. Next, at a second step, the operator inserts an insertion portion shape detection probe of the magnetic sensor system into a treatment instrument channel, and grasps an insertion portion shape three-dimensionally.

When the insertion portion shape is a shape like the insertion portion shape 173 in FIG. 32, at a third step, the operator modifies the insertion portion shape so as to become the insertion portion shape 172 in FIG. 32, and delineates the ultrasound image of the objective area again. At the next fourth step, when the insertion portion shape of the EUS is in the state of being comparatively linearized like the insertion portion shape 172 and the objective area can be delineated in the ultrasound image, the operator extracts the insertion portion shape detection probe from the treatment instrument channel.

At the next fifth step, with paying attention so as not to change the shape of the insertion portion, the operator inserts a puncture needle through the treatment instrument channel, and performs an EUS-guided puncture.

In addition, it is also possible to adopt a mechanism of changing hardness of the insertion portion as means to modify the insertion portion shape of the EUS at the third step. Such a hardness changeable mechanism is described in Japanese Patent Application Laid-Open Publication No. 2003-111717, Japanese Patent Application Laid-Open Publication No. 2001-37704, Japanese Patent Application Laid-Open Publication No. 5-168586, and the like in full detail.

When an EUS including such a hardness changeable mechanism is used in an insertion portion, at the third step, the insertion portion is linearized by raising the hardness of the insertion portion. Thereby, it is possible to obtain a linear insertion shape like the insertion portion shape 172. In addition, in the case of inserting an EUS with searching an objective area by an ultrasound scan, it enhances flexibility of an insertion portion. Thereby, both of good insert performance and exact puncture performance can be obtained.

When grasping the insertion portion shape of the EUS exactly three-dimensionally and performing linearization as much as possible at the third step, even when receiving a reaction force at the time of a puncture, the distal end portion of the EUS is less liable to retreat.

In addition, if the distal end portion of the EUS does not retreat, while puncturing, it is possible to obtain a good ultrasound image and to perform a puncture in a short time. In addition, according to the above-described method, a grasp of a two-dimensional shape using X-rays is unnecessary, and there is also no possibility of exposure.

Thus, by making the insertion shape into a linear shape, a puncture into an objective area can be performed in a shorter time, and it becomes possible to shift to a subsequent treatment quickly. This leads to shortening of procedure time and an operator's burden and a patient's pain can be reduced.

By the way, in the above description, the insertion portion shape detection probe is inserted into the treatment instrument channel at the second step. On the other hand, a trouble of inserting an insertion portion shape detection probe can be saved by providing an insertion portion shape detection mechanism in the EUS itself.

FIG. 33 is an explanatory diagram showing an EUS which has such an insertion portion shape detection mechanism.

A distal end rigid portion 182 is provided in a distal end of an insertion portion of an EUS 181. An illumination optical system, an objective optical system, and the like which are not shown are provided in the distal end rigid portion 182. In a further distal end side than the distal end rigid portion 182, an ultrasound transducer 184 is installed inside the housing 183. An ultrasound cable 187 is wired to the ultrasound transducer 184, and the proximal end side of the ultrasound cable 187 is covered with a shield 188 and an insulating tube 189.

A treatment instrument channel 185 is provided in the distal end rigid portion 182, and the treatment instrument channel 185 is extended to a channel opening portion 186. A channel tube 192 is connected to a proximal end side of the treatment instrument channel 185 through a channel ferrule 191.

Sensor coils 193-1, 193-2 . . . are arranged with keeping adequate spaces from near a distal end of the channel tube 192 so as to cover the channel tube 192. A signal cable which is not shown is wired to each of the sensor coils 193-1, 193-2 . . . , and is connected to a shape detecting apparatus which is not shown. Details of the shape detecting apparatus are disclosed in Japanese Patent Application Laid-Open Publication No. 9-28662 and Japanese Patent Application Laid-Open Publication No. 2001-46318. The shape detecting apparatus can detect a shape of an insertion portion on the basis of signals from the sensor coils 193-1, 193-2 . . . .

In addition, a shield 188 is provided in a portion of the ultrasound cable which runs side by side with the sensor coils 193-1, 193-2 . . . . Thereby, electrical noise (electromagnetic waves) arising by ultrasound transmission and reception does not mix in the sensor coils 193-1, 193-2 . . . , and does not lower position detection capability.

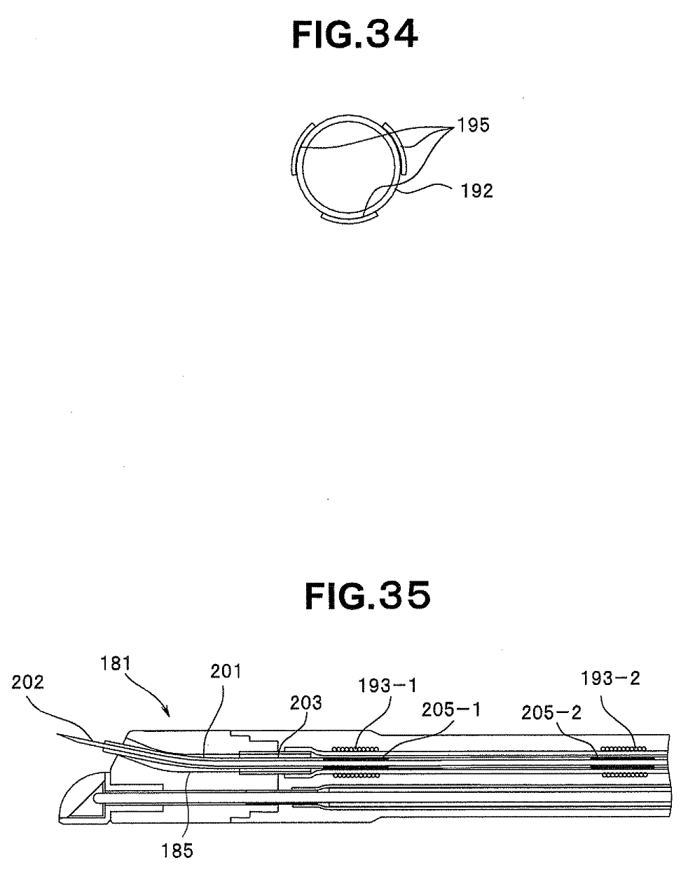

Furthermore, a plurality of strain gages 195 shown in FIG. 34 may be provided instead of the sensor coils 193-1, 193-2 . . . . FIG. 34 is an explanatory diagram for describing arrangement of strain gages. FIG. 34 shows an example of providing the strain gages 195 in three places of a circumference of the channel tube 192. The strain gages 195 can detect elongation conditions (bending state) of the channel tube 192. It is possible to detect an insertion portion shape from the elongation conditions of the channel tube 192.

Moreover, in order to detect a bend of the channel tube 192, it is good to install at least three pieces of strain gages 195 equivalently on a circumference in the same location in an axial direction. It is possible to enhance detection accuracy by increasing the number of the strain gages 195 arranged in the same location in the axial direction.

In addition, an insertion portion shape determination portion which compares angle information of a distal end side and a proximal end side may be provided in the shape detecting apparatus. When an angle of the distal end rigid portion 182 of the insertion portion of the EUS 181 and the proximal end which is not shown is 90 degrees or more according to the insertion portion shape determination portion, the shape detecting apparatus may present an operator alarm display or a beep sound.

It is possible to omit time and effort of inserting the insertion portion shape detection probe into the treatment instrument channel at the second step by adopting the EUS which has such the insertion portion shape detection mechanism.

In addition, when the insertion portion shape determination portion is provided in the shape detecting apparatus, it is also possible to judge necessity for correction of an insertion portion shape by the alarm display or beep sound on the basis of its determination. In this case, since what is necessary is to modify an insertion portion shape only when the alarm display or beep sound is given, a judgment of necessity of correction of an insertion portion shape can be made simply and promptly, thereby offering excellent working efficiency. Thereby, it is possible to shorten procedure time.

Furthermore, in order to enhance insertion portion shape detection capability, a puncture needle 201 shown in FIG. 35 is also adoptable. FIG. 35 is an explanatory diagram showing an ultrasound endoscope which adopts another puncture needle. The puncture needle 201 shown in FIG. 35 has a needle tube 202 and a sheath 203, which contains the needle tube 202 slidably, in the insertion portion.

In the sheath 203, metal pipes 205-1, 205-2 . . . which are made of ferromagnetic substance, such as iron or nickel, are press-fit and fixed in locations which approximately coincide with the sensor coils 193-1, 193-2 . . . respectively when the sheath 203 is inserted into the treatment instrument channel 185 and an operating handle in a proximal end side which is not shown is fixed to the EUS.

According to such a configuration, at the second step mentioned above, the puncture needle 201 in FIG. 35 is inserted into the treatment instrument channel 185 instead of the insertion portion shape detection probe. The detection capability of a magnetic field by the respective coils 193-1, 193-2 . . . is improved by the ferromagnetic substance of metal pipes 205-1, 205-2 . . . provided in the sheath 203 of the puncture needle 201. Thereby, it is possible to detect and display the insertion portion shape more correctly. In addition, position detection is possible even in a weaker magnetic field and an advantageous effect that power consumption can be reduced is also expectable.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. An ultrasound observation method, comprising:
locating a puncture needle in a scan area of a first ultrasound image and delineating an image of the puncture needle on the first ultrasound image;
inserting an ultrasound probe in the puncture needle; and
driving the ultrasound probe to delineate a second ultrasound image.

2. The ultrasound observation method according to claim 1, comprising protruding an ultrasound transducer of the ultrasound probe from the puncture needle at the time of insertion of the ultrasound probe into the puncture needle, and delineating the vicinity of a distal end of the ultrasound probe on the first ultrasound image.

3. The ultrasound observation method according to claim 1, wherein delineation of the second ultrasound image is to delineate a radial image, which has a section orthogonal to an insertion axis of the puncture needle, as the second ultrasound image by making the ultrasound transducer of the ultrasound probe perform an ultrasound scanning with rotating the ultrasound transducer around the insertion axis of the puncture needle.

4. The ultrasound observation method according to claim 1, wherein acquisition of the second ultrasound image is performed by setting an ultrasound frequency of the ultrasound probe at a frequency higher than an ultrasound frequency for acquiring the first ultrasound image.

5. The ultrasound observation method according to claim 1, wherein delineation of the second ultrasound image is performed by an ultrasound being emitted from the ultrasound probe, transmitting through the puncture needle, and being transmitted and received, in the state of the ultrasound transducer of the ultrasound probe being contained in the puncture needle.

6. An ultrasound endoscope system, comprising:
a first ultrasound observation unit which has a predetermined observation region;
a first ultrasound image generating unit which can display a first ultrasound image on the basis of an observation result which is observed in the first ultrasound observation unit;
a guide member with a needle-like distal end insertable and extractable to the observation region of the first ultrasound observation unit;
a second ultrasound observation unit which has an external diameter insert-through-capable to the guide member; and
a second ultrasound image generating unit which can display a second ultrasound image on the basis of an observation result which is observed by the second ultrasound observation unit.

7. The ultrasound endoscope system according to claim 6, wherein the second ultrasound observation unit includes a first ultrasound probe which can be inserted through the guide member; and
wherein the ultrasound transducer of the first ultrasound probe is arranged in the vicinity of a distal end of the guide member and performs an ultrasound scanning.

8. The ultrasound endoscope system according to claim 6, wherein the guide member has an ultrasound reflection portion in its distal end side.

9. The ultrasound endoscope system according to claim 7, wherein the first ultrasound probe makes it possible to obtain a radial image, which has a section orthogonal to an insertion axis of the guide member, as the second ultrasound image by making the ultrasound transducer perform an ultrasound scanning with rotating the ultrasound transducer around the insertion axis of the guide member.

10. The ultrasound endoscope system according to claim 6, wherein the second ultrasound observation unit performs ultrasonic observation at an ultrasound frequency higher than an ultrasound frequency of the first ultrasound observation unit.

11. The ultrasound endoscope system according to claim 6, comprising a stylet which has a toughness sensor at its distal end and can be inserted through the guide member.

12. The ultrasound endoscope system according to claim 11, wherein the guide member has an ultrasound reflection portion in its distal end side.

13. The ultrasound endoscope system according to claim 11, comprising a display unit which displays toughness information based on a detection result of the toughness sensor.

14. The ultrasound endoscope system according to claim 13, wherein the display unit displays the first ultrasound image and the toughness information on a same display screen.

15. The ultrasound endoscope system according to claim 14, wherein the display unit displays the toughness information on the same display screen in real lime with making the toughness information correspond to a motion of an image of the guide member delineated in the first ultrasound image.

16. An ultrasound endoscope system, comprising:
a first ultrasound observation unit which has a predetermined observation region;
a protruding portion which is provided with predetermined positional relation to the first ultrasound observation unit and which protrudes by a predetermined amount;
a puncture needle insertable and extractable to the predetermined observation region of the first ultrasound observation unit;
an ultrasound probe which is insertable and extractable to the first ultrasound observation unit and the predetermined observation region, and which has an ultrasound observation surface in which the protruding portion can scan; and
an ultrasound image generating unit which can display a first ultrasound image on the basis of an observation result of the first ultrasound observation unit, and a second ultrasound image on the basis of an observation result of the ultrasound probe.

17. An ultrasound endoscope system, comprising:
a first ultrasound observation unit which is provided in a distal end surface of an insertion portion of an ultrasound endoscope and which has a predetermined observation region;
a first treatment instrument channel through which a puncture needle is inserted, the first treatment instrument channel having a first opening in the distal end surface of the insertion portion;
a second treatment instrument channel having a second opening in the distal end surface of the insertion portion;
an ultrasound probe which is inserted through the second treatment instrument channel and which protrudes from the second opening;
a protruding portion which protrudes from the distal end surface of the insertion portion to a scan area of the ultrasound probe; and
an ultrasound image generating unit which can display a first ultrasound image on the basis of an observation result of the first ultrasound observation unit, and a second ultrasound image on the basis of an observation result of the ultrasound probe.

18. The ultrasound endoscope system according to claim 17, wherein the protruding portion is configured by the first ultrasound observation unit.

19. The ultrasound endoscope system according to claim 17, wherein the ultrasound image generating unit obtains a position of an image of the protruding portion, which is delineated in the second ultrasound image, in the second ultrasound image by image recognition processing, and makes a vertical direction of the second ultrasound image coincide with a vertical direction of the distal end surface of the insertion portion on the basis of the obtained position.

20. The ultrasound endoscope system according to claim 18, wherein the ultrasound image generating unit obtains a position of an image of the protruding portion, which is delineated in the second ultrasound image, in the second ultrasound image by image recognition processing, and makes a vertical direction of the second ultrasound image coincide with a vertical direction of the distal end surface of the insertion portion on the basis of the obtained position.

21. The ultrasound endoscope system according to claim 17, comprising a third treatment instrument channel which has a third opening in the distal end surface of the insertion portion.
